(19) United States
(12) Patent Application Publication
(10) Pub. No.: US 2014/0046401 A1
(43) Pub. Date: Feb. 13, 2014

(54) RETINAL PROSTHESIS

(71) Applicant: Industrial Technology Research Institute, Hsinchu (TW)

(72) Inventors: Jyh-Chern CHEN, New Taipei City (TW); Mei-Chih WANG, Toufen Township (TW); Wan-Shiun LOU, Taichung City (TW); Shih-Heng TSENG, Chiayi City (TW); Kuo-Yao WENG, Hsinchu City (TW)

(73) Assignee: INDUSTRIAL TECHNOLOGY RESEARCH INSTITUTE, Hsinchu (TW)

(21) Appl. No.: 13/950,829

(22) Filed: Jul. 25, 2013

Related U.S. Application Data
(60) Provisional application No. 61/681,985, filed on Aug. 10, 2012.

(51) Int. Cl. A61N 1/05

(52) U.S. Cl. CPC A61N 1/0543
USPC 607/54

(57) ABSTRACT

A retinal prosthesis including a microelectrode array, a polymer layer and a layer of bioactive molecule is provided. The microelectrode includes a plurality of microelectrodes. The polymer layer partly encapsulates the microelectrode array, in which the microelectrodes are exposed on the surface of the polymer layer. The layer of bioactive molecules is immobilized on the surface of the microelectrode.

### CROSS REFERENCE TO RELATED APPLICATIONS

This application claims the benefit of U.S. Provisional Application No. 61/681,985, filed Aug. 10, 2012, which is incorporated herein by reference.

### TECHNICAL FIELD

The technical field relates to a retinal prosthesis.

### BACKGROUND

The retina is a multilayered structure of different cells that is located at the inner concave surface of the back of the eye. As light enters the eye, it activates the photoreceptor cells within the retina. Light signals are transferred to electrochemical signals and conveyed to retinal neurons. The retinal neurons, in turn, relay the signals to the visual centers of the brain through the optic nerve, thereby allowing the brain to perceive visual images.

There are two shapes of photoreceptor cells in the retina, rod cells and cone cells. The rod cell contains a type of photopigment (i.e. rhodopsin) that is activated by a single photon and thus allows vision under dark conditions. The cone cell contains several types of photopigments which is activated by tens to hundreds of photons and play an important role in color vision. Various diseases or conditions may destroy the photoreceptor cell and therefore result in a partial or full vision loss. The age-related macular degeneration (AMD) and the retinitis pigmentosa (RP) are two major diseases of the retina. As the leading cause of vision loss and blindness in older adults, AMD causes both rod and cone photoreceptor cells, located within the macula at the center of the retina, to deteriorate. Furthermore, AMD affects central vision and thus causes difficulty with reading and driving. RP is an inherited condition in which the rod photoreceptor cells degenerate, causing vision loss and blindness. The loss of rod cells impairs the ability to see in the dark and gradually reduces peripheral vision until a patient suffers from tunnel vision and, ultimately, blindness.

Recent developments in the field of retinal prostheses have proceeded at a breathtaking rate and offer great promise to restore the vision of blind patients with RP or AMD. Retinal prostheses function by producing stimulating currents through a microelectrode array that alters the membrane potential of adjacent retinal neurons. Retinal prostheses so far can be divided into two types: those that receive power from incident light acting through passive or active photodiodes, and those that receive power and information based on cables or transcutaneous telemetry systems. Retinal prosthetic devices can also be categorized according to the site of implantation of the microelectrode array into epiretinal and subretinal prostheses.

The epiretinal prosthesis provides electrical stimulation to ganglion cells of the retina, through a microelectrode array implanted at the epiretinal space. The retina is covered by an inner limiting membrane. One major disadvantage with the epiretinal prosthesis is that the inner limiting membrane has high impedance to electrical stimulation. Thus, the epiretinal prosthesis requires an over ten times higher charge at the electrode surface, to achieve the same stimulation level on the target ganglion cells, when compared to central nervous system stimulation and subretinal stimulation.

The subretinal prosthesis installs the microelectrode array in the subretinal space and behind the retina, thus avoiding the high-impedance barrier problem. The microelectrode array is very close to the bipolar cells, allowing lower charge stimulation. A subretinal prosthesis requires that all components be fitted behind the retina, with circuits integrated with the microelectrode array. Power can be transferred into the subretinal prosthesis by incident light, cables or transcutaneous telemetry systems. The power output needs to be strictly controlled, since heat dissipation is limited in the subretinal space, and overheating can easily damage the retina.

The spatial resolution of retinal prostheses is affected by the number, size, spacing and distance to target cells of the microelectrodes within an array. It has been investigated that a microelectrode array of 60 electrodes is the minimum requirement for spot reading or object recognition, and a microelectrode array of 1000 electrodes is required for an appropriate visual acuity of 20/200 for AMD patients.

The microelectrode array of the epiretinal prostheses is separated by the inner limiting membrane and nerve fiber layer. For the case of a subretinal prosthesis, the microelectrode is separated by the photoreceptor remnants or subretinal scar tissue. Cross-talk between adjacent microelectrodes will occur due to a larger distance between the microelectrodes and target neural cells. It will also increase the charge density and power for cell stimulation, which in turn, will result in erosion of electrodes and excessive resistive heating of tissues. Furthermore, encapsulation with fibrous tissues may occur with any implanted retinal prosthesis, including a retinal pigment epithelium reaction to a subretinal prosthesis. Living cells that grow on the device's surface may lead to unknown chemical or biological environments which may trigger or accelerate degradation processes of the device. Such reactions may also disrupt contact between the microelectrode array and target ganglion cells or bipolar cells. Moreover, it must take into account that retinal neural cells are not aligned in two-dimensional layers. The variability of distance between microelectrodes and cells in different parts of the implant will result in strong variations of the stimulation signal, and therefore may not achieve optimal contrast or pattern recognition.

### SUMMARY

One embodiment of the disclosure provides a retinal prosthesis comprising a microelectrode array comprising a plurality of microelectrodes, a polymer layer partly encapsulating the microelectrode array, wherein the microelectrodes are exposed on the surface of the polymer layer, and a layer of bioactive molecules immobilized on the surface of the microelectrode.

One embodiment of the disclosure further provides a method for the treatment of degenerative retinal diseases comprising applying the retinal prosthesis to the retina of a subject in need.

A detailed description is given in the following embodiments with reference to the accompanying drawings.

### BRIEF DESCRIPTION OF THE DRAWINGS

The embodiments can be more fully understood by reading the subsequent detailed description and examples with references made to the accompanying drawings, wherein:

FIG. 1A˜1M′ show one embodiment of the disclosure for making an epiretinal prosthesis with a bio-functionalized and parylene-based microelectrode array;

FIG. 2A˜2M′ show one embodiment of the disclosure for making an epiretinal prosthesis with a bio-functionalized and polyimide-based microelectrode array;

FIG. 3A˜3M′ show one embodiment of the disclosure for making a subretinal prosthesis with a bio-functionalized and parylene-based microelectrode array;

FIG. 4 shows the fluorescent images of immobilized bioactive molecules, laminin and L1CAM, on the surface of parylene-C substrate by the immune-fluorescence staining method according to one embodiment;

FIG. 5A shows cell viability of C6 glial cells on a parylene-C substrate with a layer of L1CAM, laminin or collagen on the surface of the substrate according to one embodiment;

FIG. 5B shows the immobilization of C6 glial cells on a parylene-C substrate with a layer of L1CAM, laminin or collagen on the surface of the substrate by the analyses of glial fibrillary acidic protein (GFAP) and DAPI according to one embodiment;

FIG. 6A shows the attachment of NGF-primed PC12 neural cells on parylene-C substrates with a layer of L1CAM, laminin or collagen on the surface of the substrate according to one embodiment.

FIG. 6B shows that the outgrowth of NGF-primed PC12 neural cells on parylene-C substrates with a layer of L1CAM, laminin or collagen on the surface of the substrate according to one embodiment.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

Artificial materials implanted in the neural tissue have a tendency to induce glial scar formation. This glial scar formation mainly results from glial cells such as astrocytes and microglia. The scar tissue nearby the implanted devices will damage the performance of the devices resulting in a decrease in the local density of neurons and increase of electrode impedance. In addition, the distance between target neural cells and microelectrodes limits the number of microelectrodes and the ability to stimulate specific types of retinal neural cells that contribute to visual perceptions. Further, it has been found that the neural cell density around the microelectrode is significantly lower than the bulk neural cell density. Therefore, the formation of gliosis poses one of the greatest challenges in the field of retinal prostheses, which require a higher density of microelectrodes in a stimulating array and spatial resolution.

One embodiment of the present disclosure is to provide a retinal prosthesis for improving the spatial resolution by immobilizing a layer of bioactive molecules on a microelectrode array of the retinal prosthesis to form a bio-functionalized microelectrode array. Due to the layer of bioactive molecules, the biofunctionalized microelectrode array is able to selectively promote the attachment of neural cells onto the microelectrode array and neurite outgrowth. For instance, the layer of bioactive molecules immobilized onto the array according to the disclosure is able to inhibit the attachment and cell growth of glial cells which involve gliosis formation to the array, while it promotes the attachment and outgrowth of NGF-primed PC12 neural cells which do not lead to gliosis formation. Therefore, the retinal prosthesis according to the embodiments of the present disclosure is capable of simultaneously inhibiting the formation of gliosis. In addition, according to embodiments of the disclosure, the distance between the neural cells and the microelectrodes is minimized and, therefore, the crosstalk of adjacent microelectrodes of a retinal prosthesis can be reduced. The retinal prosthesis according to the disclosure is able to bring neural cells into close proximity to the microelectrodes so that the electrical power required for neural cell excitation is reduced and tissue heating and electrode erosion are decreased. Therefore, the retinal prosthesis according to embodiments of the disclosure allows for a higher density of microelectrodes so as to provide improved spatial resolution. Another embodiment of the present disclosure is to provide a method for the treatment of degenerative retinal diseases, such as glaucoma, retinitis pigmentosa (RP) or age-related macular degeneration (AMD), by using the retinal prosthesis described above.

According to embodiments of the disclosure, a retinal prosthesis comprising a microelectrode array including a plurality of microelectrodes, a polymer layer partly encapsulating the microelectrode array, wherein the microelectrodes are exposed on the surface of the polymer layer, and a layer of bioactive molecules, immobilized on the surface of the microelectrode. The layer of bioactive molecules may further be immobilized on the surface of the polymer layer.

The bioactive molecule according to embodiments of the disclosure comprise L1 cell adhension molecule (L1CAM), also named CAML1, CD171, HSAS, HSAS1, MASA, MIC5, N-CAML1, S10, SPG1 or HGNC. L1CAM is a 200-220 kDa glycoprotein and cell adhesion molecule (NCAM), a member of the immunoglobulin superfamily, which consists of six immunoglobulin-like domains, five fibronectin-like repeats (type III), and a single transmembrane sequence linked to a conserved cytoplasmic domain. It has been reported that L1CAM plays an important role in nervous system development, including neuronal migration, differentiation and guiding axons into their target area. In addition, it has been studied that L1CAM selectively promotes neural cell attachment and outgrowth in the presence of astrocytes and fibroblasts.

The “microelectrode array” recited in embodiments of the disclosure refers to a microelectrode array with a function of producing stimulating currents to alter the membrane potential of retinal neurons. The microelectrode array may be designed for epiretinal or subretinal prostheses. As designed for an epiretinal prosthesis, the microelectrode array may comprise a substrate and a plurality of microelectrodes on the surface of the substrate for sending electrical signals to stimulate retinal neural cells adjacent to the microelectrodes. As designed for subretinal prosthesis, the microelectrode array may comprise a substrate, a plurality of microelectrodes on the surface of the substrate, a plurality of photo sensors on the surface of the substrate, an integrated circuit within the substrate, and a power receiver within the substrate. The microelectrode recited herein has functions for sending electrical signals to stimulate retinal neural cells adjacent to the microelectrodes. The photo sensor used herein is for receiving the optical imaging signal and converting the optical imaging signal into the electrical signal. The integrated circuit used herein is in connecting with the microelectrodes and the photo sensors for receiving the electrical signal from each photo sensor, amplifying the electrical signal into the stimulation signal, and sending the stimulation signal to the microelectrode that is coupled with the photo sensor. The powder receiver used herein has functions for receiving energy from an external power source, transforming energy into the electrical power and sending the electrical power to the integrated circuit. However, the elements of the microelectrode array recited in the disclosure are not limited thereto. A person skilled in the art may adequately adjust the elements of the microelectrode array according to its potential use.

The microelectrode may consist of a Ti/metal double-layer or a Ti/metal/Ti triple-layer between the electrode material and the polymer layer. The metal of the Ti/metal double-layer and Ti/metal/Ti triple-layer may be a conductive material selected from a group consisting of aluminum (Al), titanium (Ti), platinum (Pt), gold (Au) and the alloy thereof, but is not limited thereto. The thickness of Ti/metal double-layers layer is preferably greater than 250 nm and less than 1 μm, in which the thickness of Ti layer is greater than 50 nm and less than 1 μm, and the thickness of metal layer is greater than 200 nm and less than 1 μm. The thickness of Ti/metal/Ti triple-layers layer is preferably greater than 300 nm and less than 1 μm, in which the thickness of Ti layer is greater than 50 nm and less than 1 μm, and the thickness of metal layer is greater than 200 nm and less than 1 μm.

For the reduction of gliosis formation on the surface of the microelectrode array, biocompatible electrode materials with neural stimulation accuracy are required. The biocompatible electrode material, such as titanium (Ti), titanium nitride (TiN), platinum (Pt) or iridium oxide (IrOx), has been reported to coated or sputtered onto the surface of the microelectrodes. IrOx is considered as a suitable material for electrical stimulation of neural cells due to a high reversible charge delivery capacity when compared with other metallic electrode material. With a high charge delivery capacity, electrical charges can be delivered to neural cells in a smaller electrode design, allowing for a higher density electrode array and increasing the stimulation resolution. Furthermore, the IrOx undergoes a pure reversible electrochemical reaction between Ir(III) and Ir(IV) oxidation states within its IrOx/Ir redox system, which means that no new substance is formed and hence no harmful reactants are released into tissues. This makes IrOx (x≦4) more effective for charge injection than other biocompatible electrode materials that are based on a surface reaction of metal oxidation.

An IrOx coating/sputtering film can be formed by activated iridium oxide films (AIROF) or sputtered iridium oxide films (SIROF). The AIROF can be made by first depositing an iridium film on a substrate by sputtering or e-beam evaporation, and then by repeated oxidation and reduction between Ir films and electrolytes during potential cycling. A hydrated oxide film can be formed on the metal surface. The SIROF can be obtained by radio frequency (RF) sputtering or direct current (dc) sputtering iridium in ambient oxygen.

In one example of the disclosure, an IrOx (x≦4) layer is sputtered on the surface of the microelectrode by SIROF and the thickness of IrOx layer is greater than 100 nm and less than 1 μm.

The microelectrode array according to embodiments of the disclosure is encapsulated within a polymer layer, such as parylene-C or polyimide. The thickness of the polymer layer according to the disclosure is preferably greater than 10 μm and less than 20 μm.

Parylene-C is known to be a uniform, pinhole-free, conformal, anticorrosive, flexible, transparent, and chemically inert insulating material with a low dielectric constant (˜3), high mechanical strength (Young's modulus ˜4 GPa) and the highest biocompatibility certification. United States Pharmacopoeia (USP) Class VI. Parylene-C has a low water and gas permeability compared with polydimethylsiloxane (PDMS) and polyimide (PI). Due to its hydrophobic property, Parylene-C has a recognized issue of having poor wet adhesion to smooth surface materials as metal and silicon wafer.

Parylene-C is synthesized from a low-molecular weight di-para-xylene dimmer using a solvent-less polymerization process. Solid powders of di-para-xylene dimmer are first vaporized at 150° C. and 100 mTorr. The second step is the pyrolysis of the dimer to yield the monomer (para-xylylene) at 680° C. and 500 mTorr. Finally, monomer gas enters a deposition chamber where it is adsorbed onto substrates to form a solid polymerized thin film at room temperature and 1.0 mTorr.

Polyimide has been used as an insulator in microelectronics because of its numerous excellent properties such as: excellent resistance to solvents, strong adhesion to metals and metal oxides and good dielectric properties. The volume resistivity and dielectric strength of polyimide are comparable to silicon and typical silicon insulation materials, such as SiO₂ and Si₃N₄. When used as a neural implant material, polyimide also has appropriate mechanical properties (Young's modulus ˜2.5 GPa) and its biocompatibility has been demonstrated in many studies in vitro and in vivo. Furthermore, a flexible, lightweight polyimide structure can reduce the mechanical stress transferred to the surrounding tissue compared to brittle silicon structures. Same as Parylene-C, Polyimide thin films are patterned using standard microfabrication processes such as photolithography and reactive ion etching. Photolithography and thin film deposition could be useful for a microelectrode array with high channel density. If used, it would result in a higher spatial resolution which is necessary for accurate stimulation of a tiny nervous structure.

### First Embodiment of the Disclosure

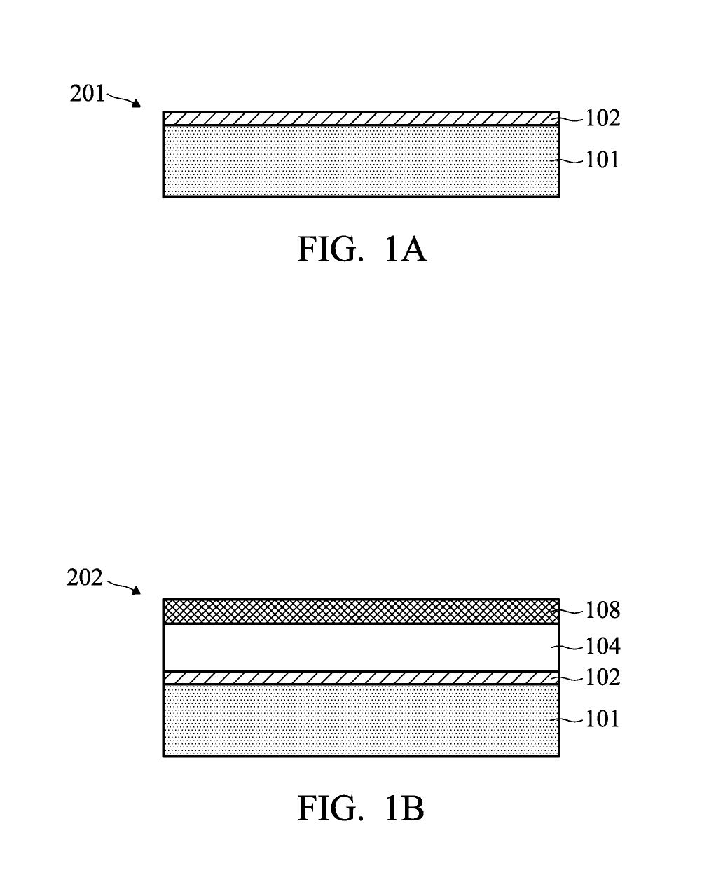

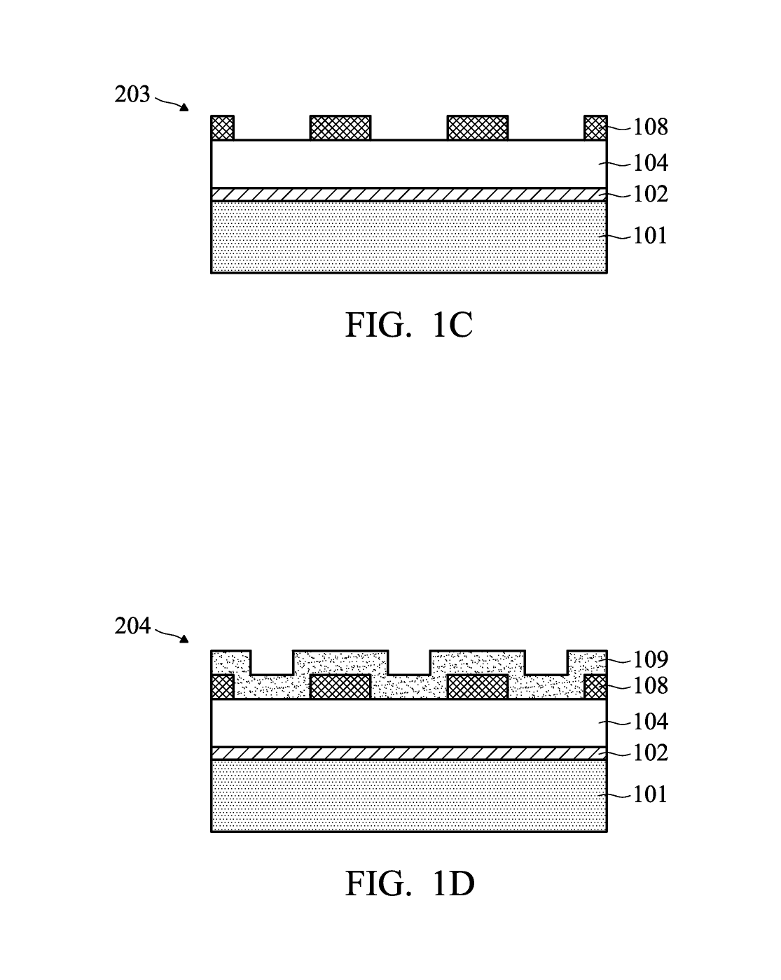

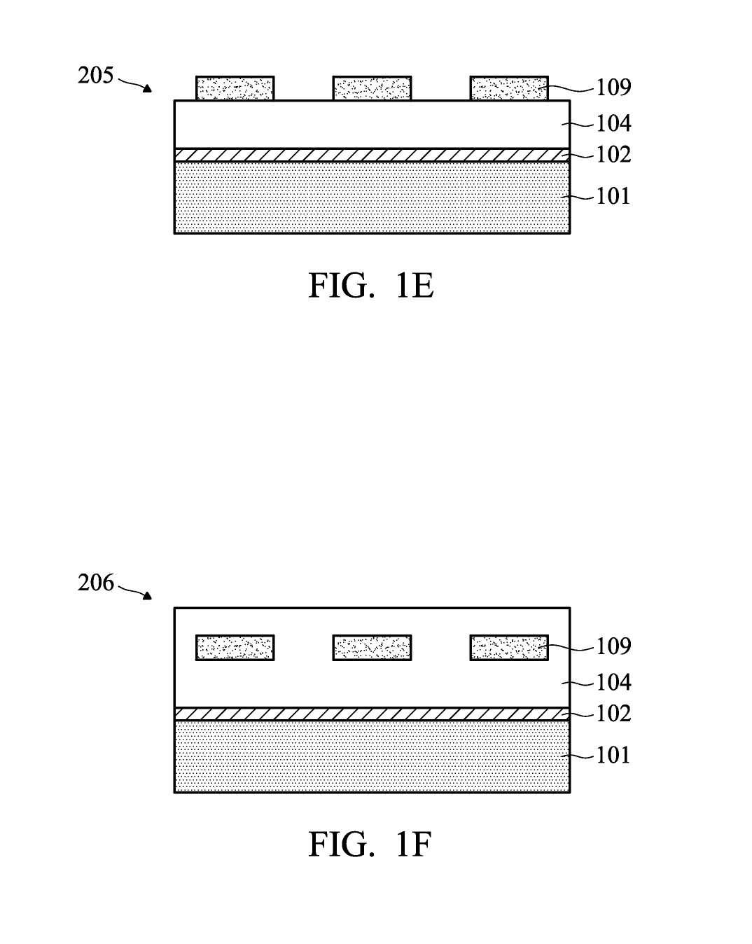

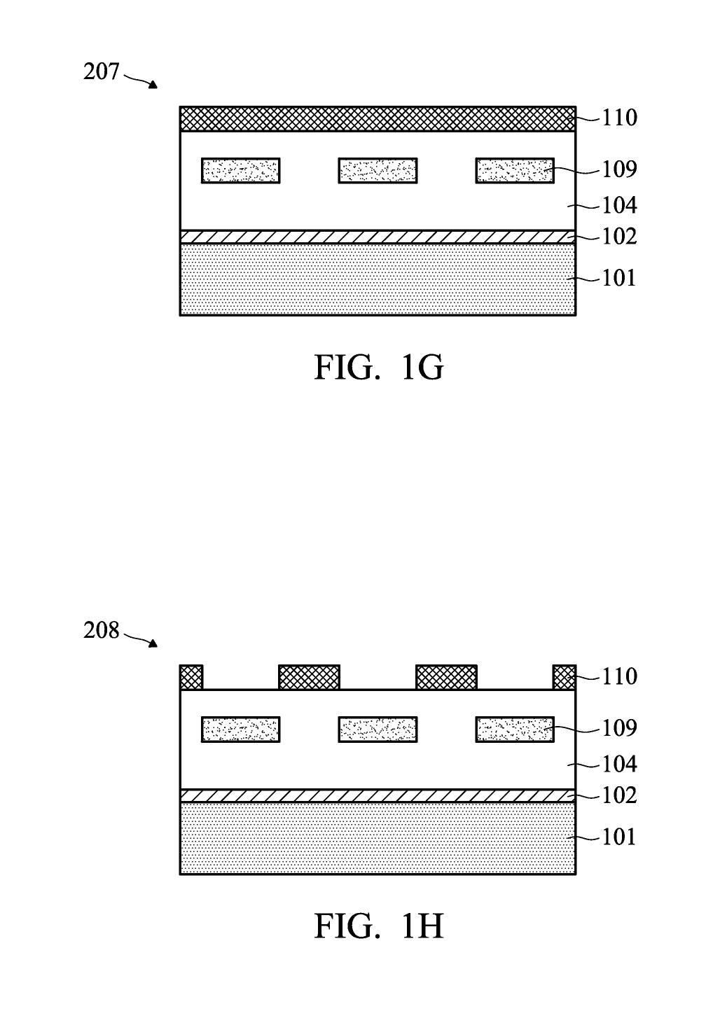

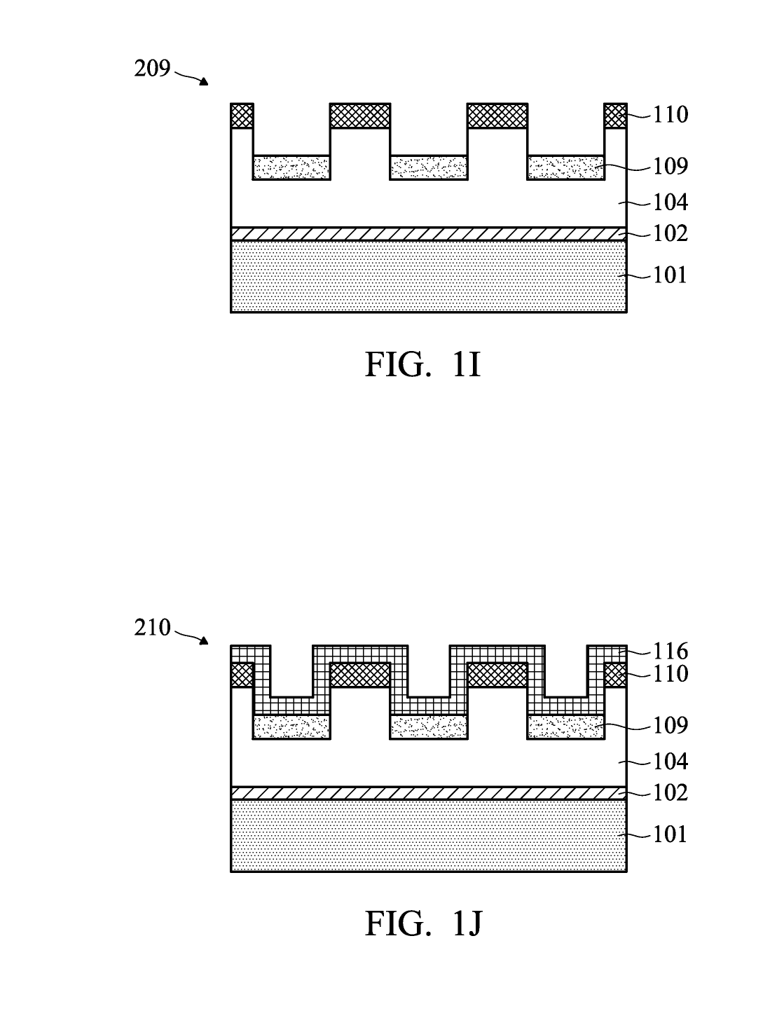

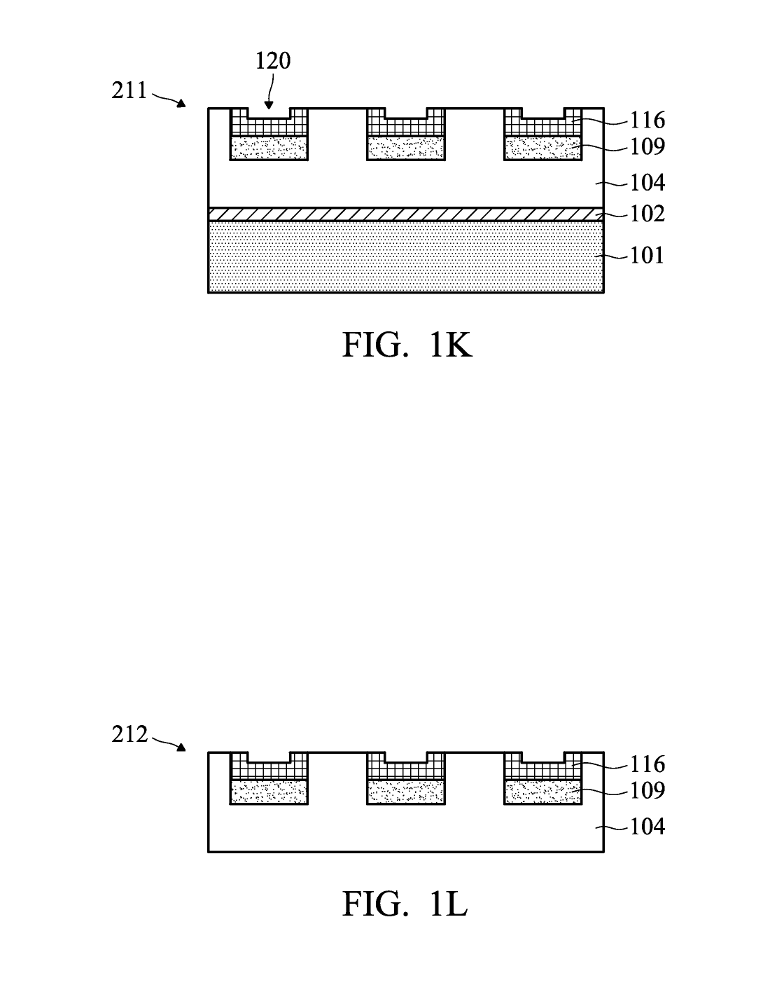

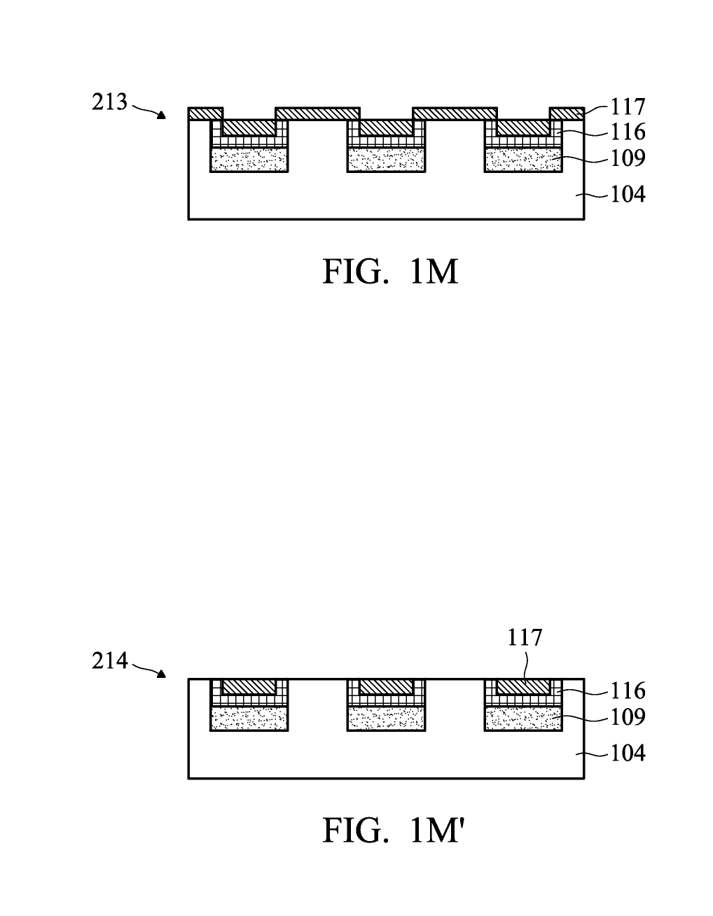

The first embodiment of the disclosure provides a process for fabricating a biofunctionalized and polymer-based microelectrode array of an epiretinal prosthesis, in which the polymer is parylene-C. As shown in FIG. 1A, a silicon wafer 101 was pre-deposited with a silicon oxide layer 102 with less than 1 μm of thickness by plasma-enhanced chemical vapor deposition (PECVD) as a starting layer in Step 201. In Step 202, a parylene-C layer 104 having 10-15 μm of thickness was deposited on the surface of silicon oxide layer 102 (FIG. 1B). Then, a sacrificial separation photoresist layer 108 was spun coated on the surface of parylene-C layer 104. The photoresist layer 108 was patterned to define the area of microelectrodes and contact pads in Step 203 (FIG. 1C). Titanium (Ti) (50-100 nm thickness), gold (400-1500 nm thickness), and titanium (50-100 nm thickness) layers were consecutively deposited on the parylene-C layer 104 in a sputter chamber with an argon (Ar) atmosphere of less than 0.005 mTorr in Step 204 (FIG. 1D). The Ti layers formed biocompatible adhesion layers between the electrode materials (Au and IrOx) and the parylene-C layer. Subsequently, the multi-layer metal of Ti/Au/Ti 109 was patterned by a metal wet etching process with a photoresist 108 as a mask in Step 205 (FIG. 1E). After the photoresist mask 108 was removed, the parylene-C layer 104 was deposited again to seal the metal structure in Step 206 (FIG. 1F). The sacrificial separation photoresist layer 110 was spun coated on the surface of parylene-C layer 104 in Step 207 (FIG. 1G). The photoresist layer 110 was then patterned to define the area of microelectrodes and contact pads in Step 208 (FIG. 1H). Thereafter, unwanted parylene-C 104 was removed using oxygen plasma in a reactive ion etch (RIE) system with a photoresist 110 as a mask in Step 209 (FIG. 1I).

An IrOx layer 116 with 300-800 nm of thickness was sputtered onto the Ti/Au/Ti layer 109 and patterned by the same photolithography process as described above in Ar/O₂ plasma using an RF magnetron sputter in Step 210 (FIG. 1J). Sputtering was performed with 1-2 second pulses followed by 5-10 second cool-down times to maintain low parylene-C substrate temperatures. For the deposition of Ti, Au and IrOx films, the target purities of Ti, Au and Ir were greater than 99.9%. The target sizes were 4 inches in diameter and 0.125 inches in thickness. Prior to sputtering, the vacuum chamber was evacuated to a pressure of less than 0.005 mTorr. A throttle valve was used to control the working pressure during sputtering. During the deposition of IrOx 116, oxygen was injected into the chamber to create Ar/O₂ gas ratios 1/1, 2/1, 4/1, 8/1 and 20/1. The Ar flow rate was fixed at 40 sccm (standard cubic centimeter per minute), and the oxygen flow rate was varied from 2 to 40 sccm. RF power was kept at 100 W. The resulting working pressure inside the vacuum chamber varied from 2-100 mTorr. Subsequently, the IrOx layer 116 was patterned by a lift-off process, using previously defined photoresist patterns in Step 211 (FIG. 1K). The opening area of the electrode sites 120 was 50×50 μm². A set of microelectrode stimulation pads with a geometric surface area of 1 mm², traces, and bonding pads on the electrode were fabricated as continuous titanium and iridium oxide layers 116 on the parylene-C layer 104. The silicon wafer 101 was removed from the parylene-C layer 104 in Step 212 (FIG. 1L).

The bioactive molecules 117 as L1CAM were immobilized on the surface of IrOx 116 in Step 213 and 214 by piezoelectric dispensing technologies, or immobilized on the surface of IrOx 116 and parylene-C 104 by direct coating (FIG. 1M and FIG. 1M′). In brief, the surface of parylene-C was first cleaned and roughened by a 200 mTorr oxygen plasma with 30 W power for 30 seconds. The droplet contact angle measurement showed that the original parylene-C had the hydrophobic characteristic of a contact angle of 90-100 degrees. After the oxygen plasma treatment, a significant drop in the contact angle was around 30 degrees. An oxygen plasma treatment also created free radicals on the polymer surface that supported subsequent covalent bonding for immobilizing bioactive molecules. Immediately after the oxygen plasma treatment, a solution of a predetermined amount of bioactive molecules 117 as L1CAM was added onto the surface of the parylene-C layer 104 and it was placed inside an oven at 37° C. for 2 hours to allow for the immobilization of bioactive molecules onto the surface of IrOx layer 116 and parylene-C layer 104. The parylene-C layer 104 and the IrOx layer 116 were washed with the phosphate buffer solution to remove unbounded bioactive molecules 117. Finally, the substrate with microelectrode arrays was then diced by using a femtosecond laser to obtain a biofunctionalized microelectrode array of epiretinal prostheses.

### Second Embodiment of the Disclosure

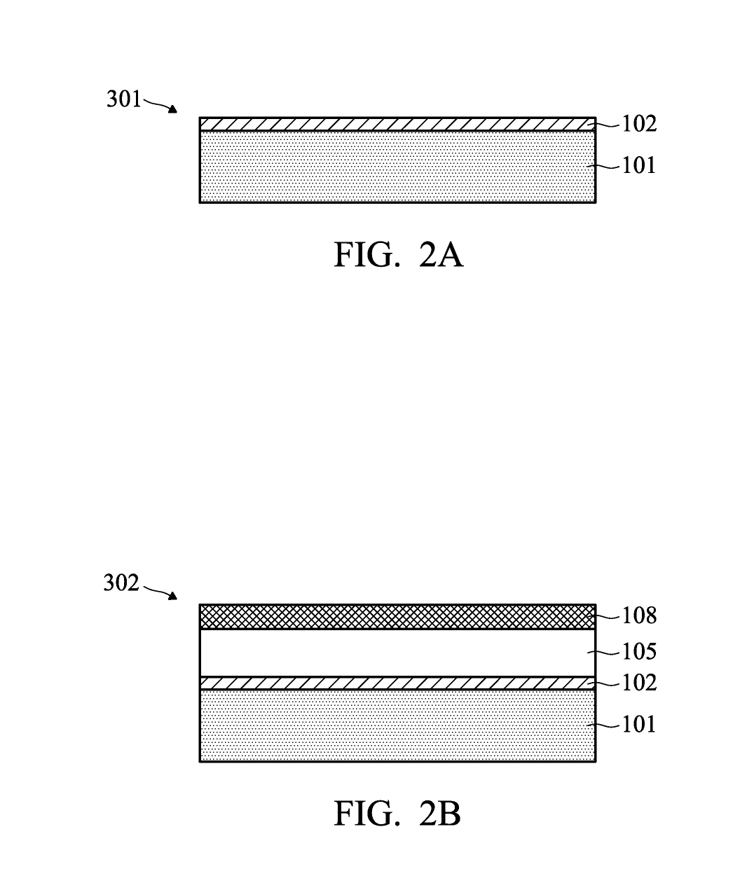

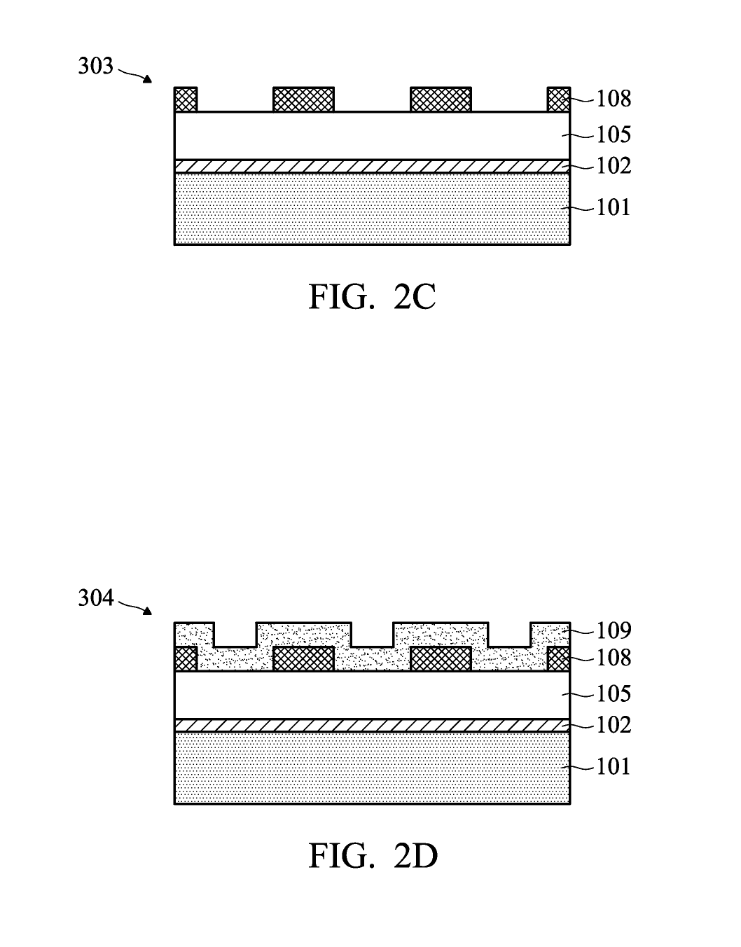

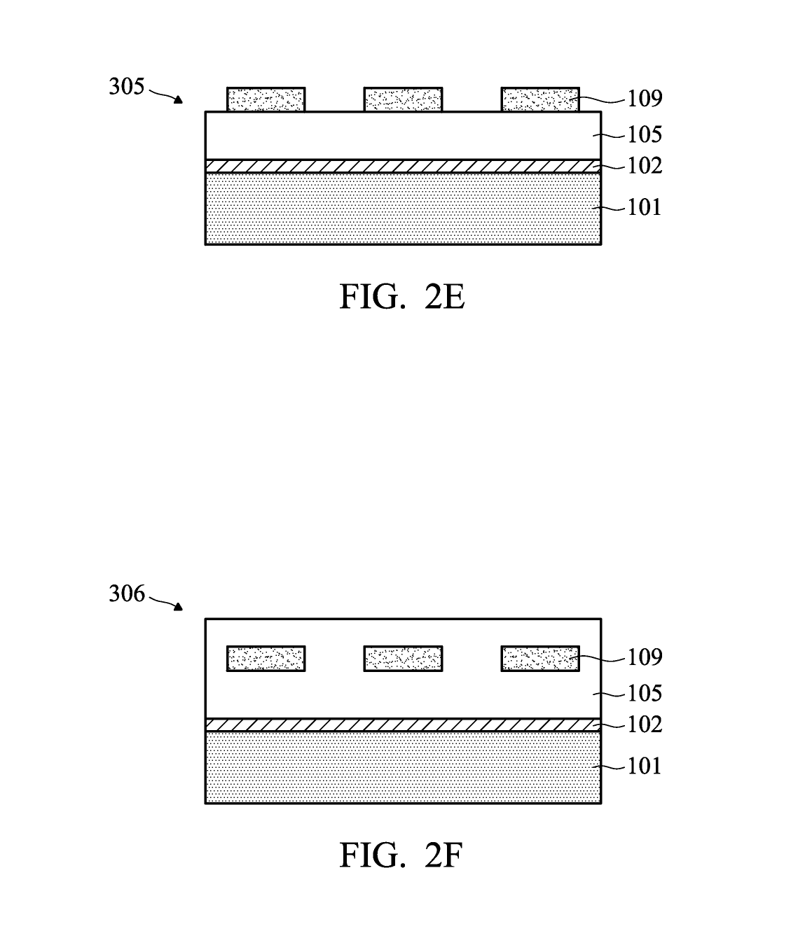

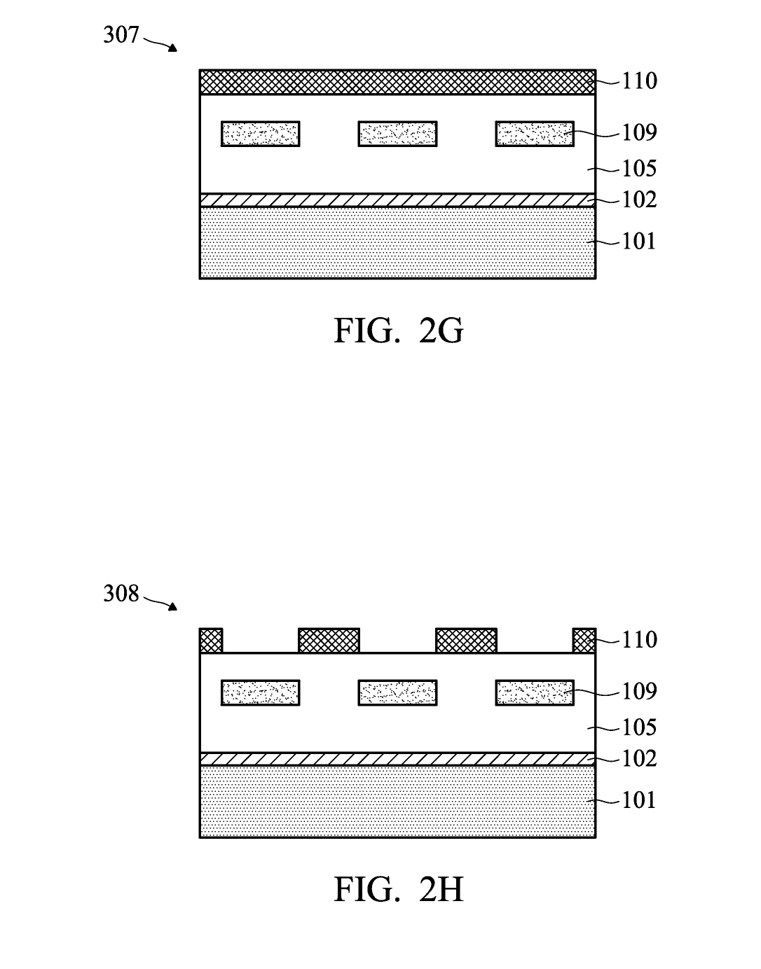

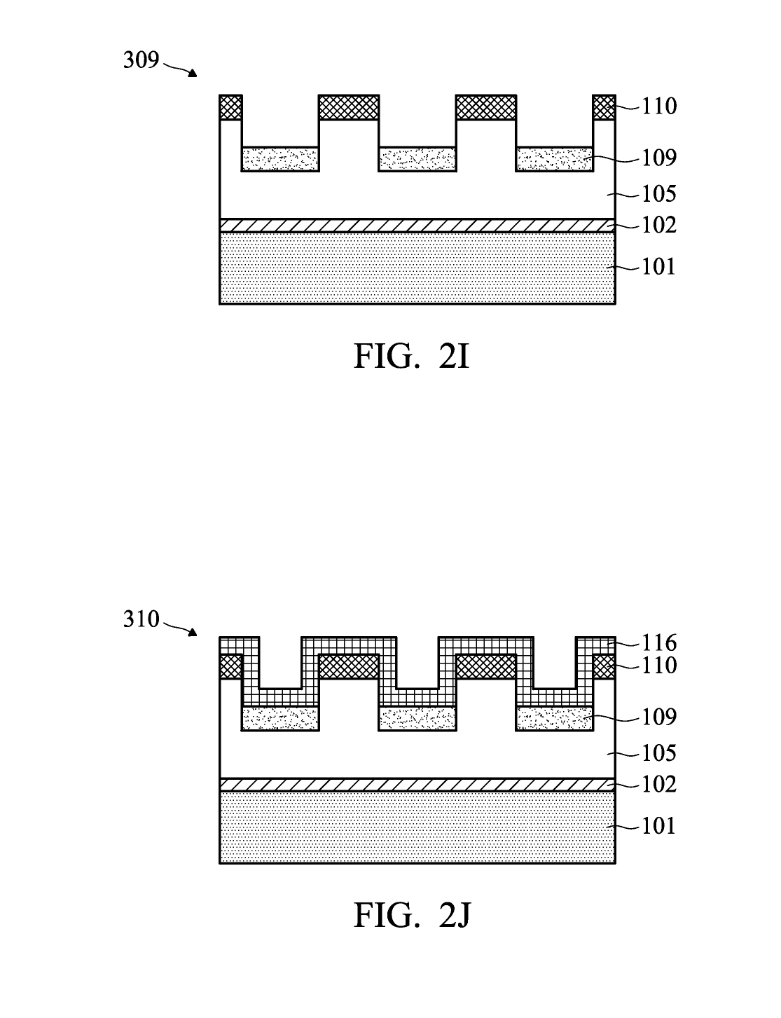

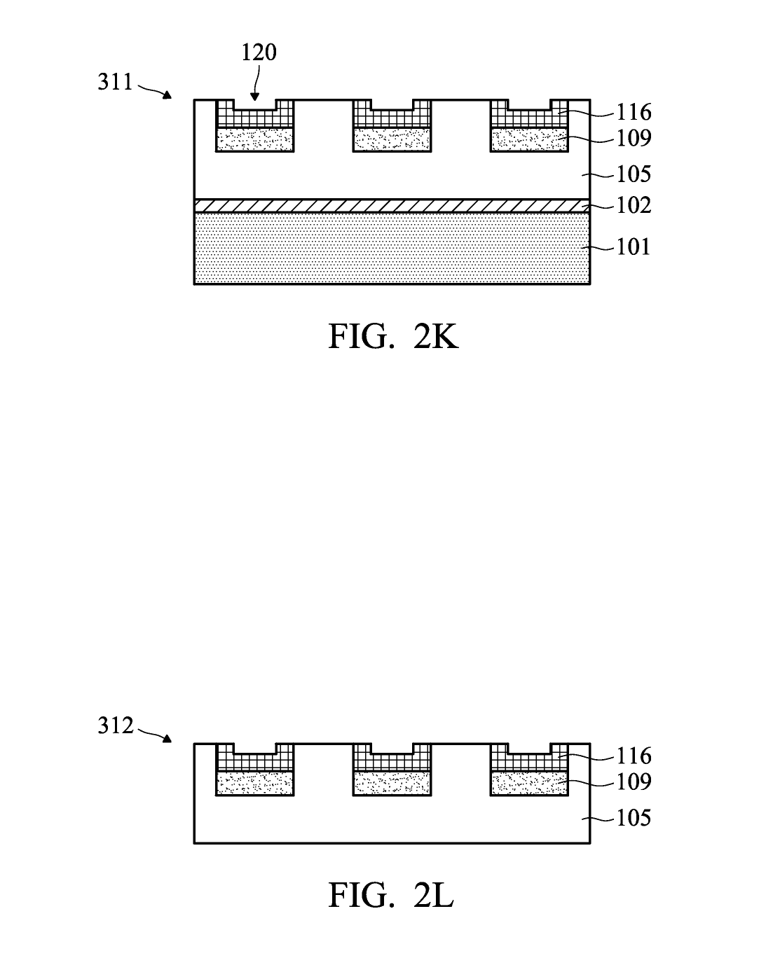

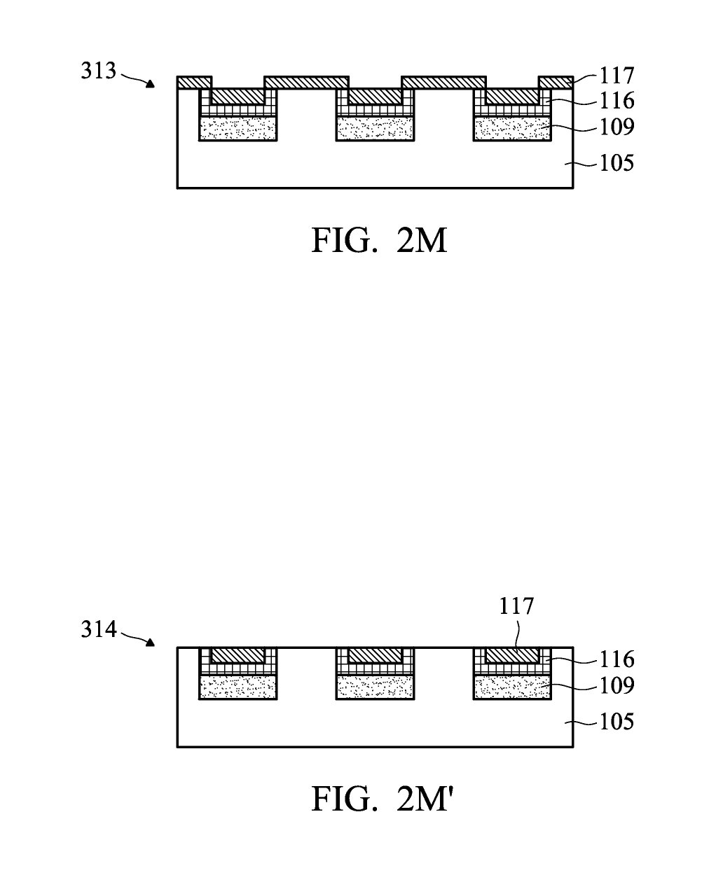

The second embodiment of the disclosure provides a process for fabricating a bio-functionalized and polymer-based microelectrode array of an epiretinal prosthesis, in which the polymer is polyimide. As shown in FIG. 2A, a silicon wafer 101 was pre-deposited with a silicon oxide layer 102 with less than 1 μm of thickness by plasma-enhanced chemical vapor deposition (PECVD) as a starting layer in Step 301. A polyimide layer 105 having 10-20 μm of thickness was spun coated on the surface of silicon oxide layer 102 in Step 302 (FIG. 2B). Then, a sacrificial separation photoresist layer 108 was spun coated on the surface of polyimide layer 105. The photoresist layer 108 was patterned to define the area of microelectrodes and contact pads in Step 303 (FIG. 2C). In Step 304, titanium (Ti) (50-100 nm thickness), gold (400-1500 nm thickness), and titanium (50-100 nm thickness) layers were consecutively deposited on the parylene-C layer 104 in a sputter chamber with an argon (Ar) atmosphere of less than 0.005 mTorr (FIG. 2D). The Ti layers formed biocompatible adhesion layers between the electrode materials (Au and IrOx) and the polyimide layer. The multi-layer metal of Ti/Au/Ti 109 was patterned by a metal wet etching process with a photoresist 108 as a mask in Step 305 (FIG. 2E). After the photoresist mask 108 was removed, the polyimide layer 105 was spin coated again to seal the metal structure in step 306 (FIG. 2F). The sacrificial separation photoresist layer 110 was spun coated on the surface of polyimide layer 105 in Step 307 (FIG. 2G). Then, the photoresist layer 110 was patterned to define the area of microelectrodes and contact pads in Step 308 (FIG. 211). Subsequently, unwanted polyimide 105 was removed using oxygen plasma in a reactive ion etch (RIE) system with a photoresist 110 as a mask in step 309 (FIG. 21).

An IrOx layer 116 with 300-800 nm of thickness was sputtered onto the Ti/Au/Ti layer 109 and patterned by the same photolithography process as described above in Ar/O₂ plasma using an RF magnetron sputter in Step 310 (FIG. 2J). Sputtering was performed with 1-2 second pulses followed by 5-10 second cool-down times to maintain low polyimide temperatures. For the deposition of Ti, Au and IrOx films, the target purities of Ti, Au and Ir were greater than 99.9%. The target sizes were 4 inches in diameter and 0.125 inches in thickness. Prior to sputtering, the vacuum chamber was evacuated to a pressure of less than 0.005 mTorr. A throttle valve was used to control the working pressure during sputtering. During the deposition of IrOx, oxygen was injected into the chamber to create Ar/O₂ gas ratios 1/1, 2/1, 4/1, 8/1 and 20/1. The Ar flow rate was fixed at 40 sccm (standard cubic centimeter per minute), and the oxygen flow rate was varied from 2 to 40 sccm. RF power was kept at 100 W. The resulting working pressure inside the vacuum chamber varied from 2-100 mTorr. The IrOx layer 116 was patterned by a lift-off process, using previously defined photoresist patterns in Step 311 (FIG. 2K). The opening area of the electrode sites 120 was 50×50 nm². A set of microelectrode stimulation pads with a geometric surface area of 1 mm², traces, and bonding pads on the electrode were fabricated as continuous titanium and iridium oxide layers on the polyimide layer 105. The silicon wafer 101 was removed from the polyimide layer 105 in Step 312 (FIG. 2L).

The bioactive molecules 117 as L1CAM were immobilized on the surface of IrOx 116 by piezoelectric dispensing technologies, or immobilized on the surface of IrOx 116 and polyimide 105 by direct coating in Step 313 and 314 (FIG. 2M and FIG. 2M′). In brief, the surface of polyimide was first cleaned and roughened by a 200 mTorr oxygen plasma with 30 W power for 30 seconds. Immediately after the oxygen plasma treatment, a solution of a predetermined amount of bioactive molecules 117 as L1CAM was added onto the surface of polyimide layer 105 and it was placed inside an oven at 37° C. for 2 hours to allow for the immobilization of bioactive molecules onto the surface of IrOx layer 116 and polyimide layer 105. The polyimide layer 105 and the IrOx layer 116 were washed with the phosphate buffer solution to remove unbounded bioactive molecules 117. Finally, the substrate with microelectrode arrays was diced by using a femtosecond laser to obtain a biofunctionalized microelectrode array of epiretinal prostheses.

### Third Embodiment of the Disclosure

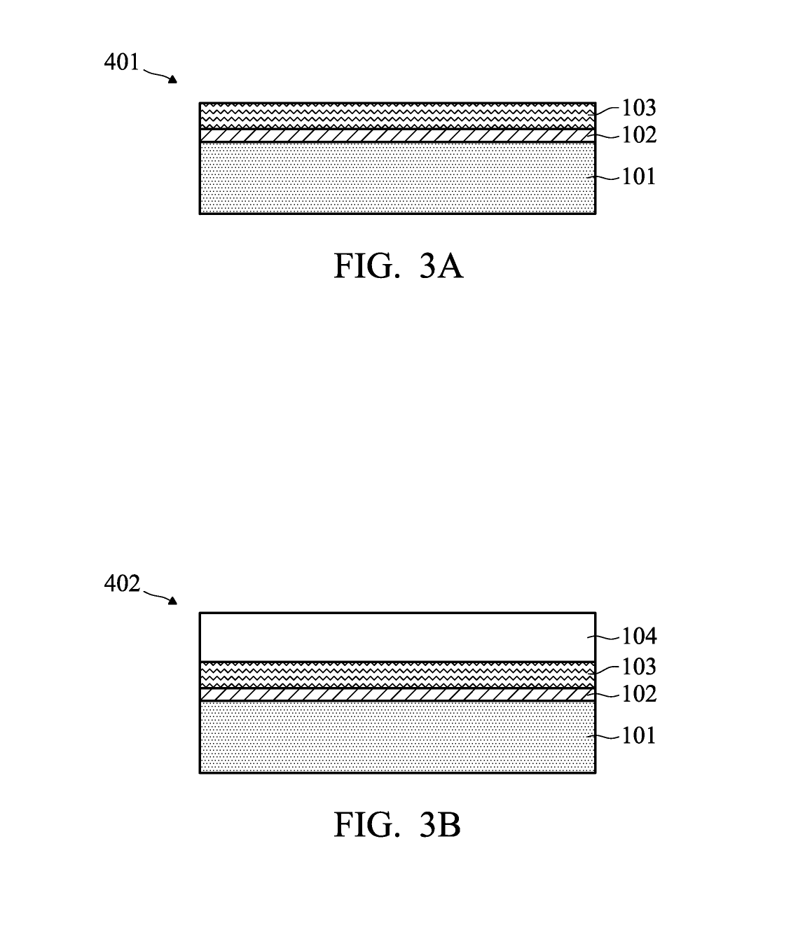

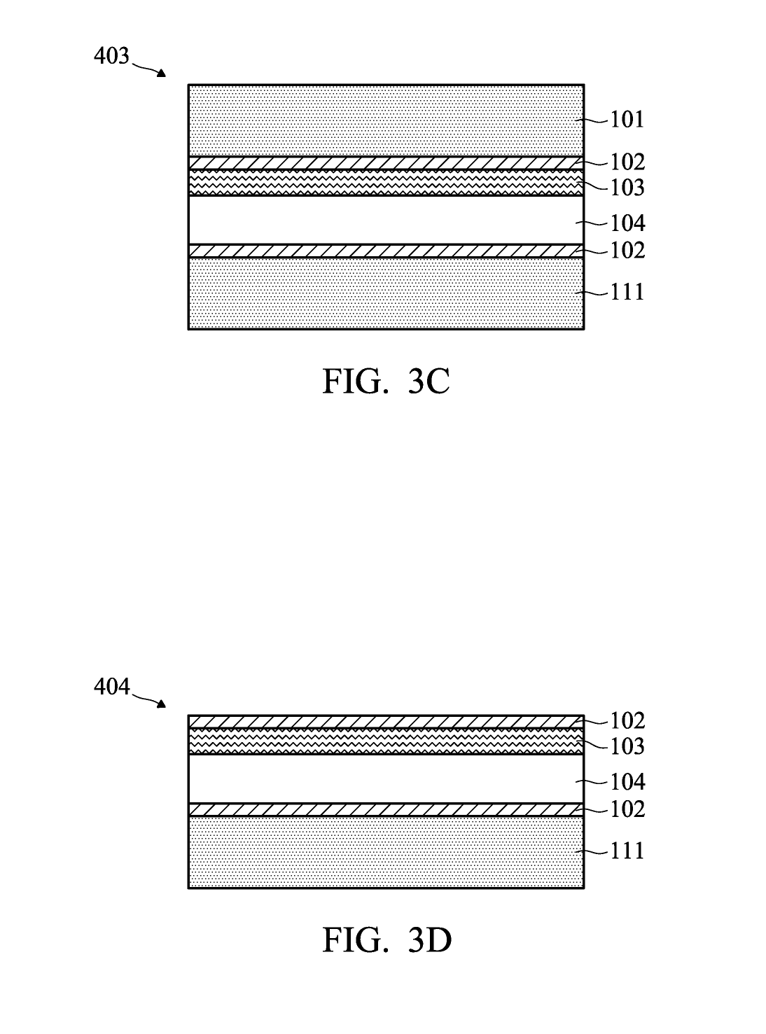

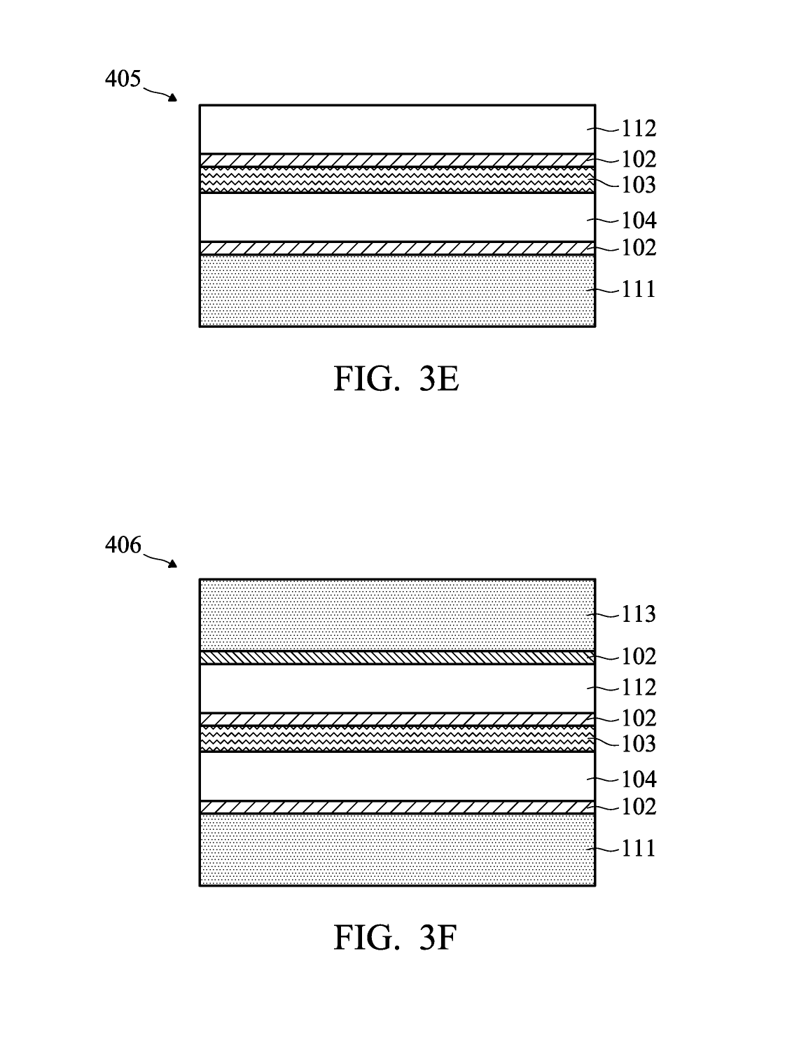

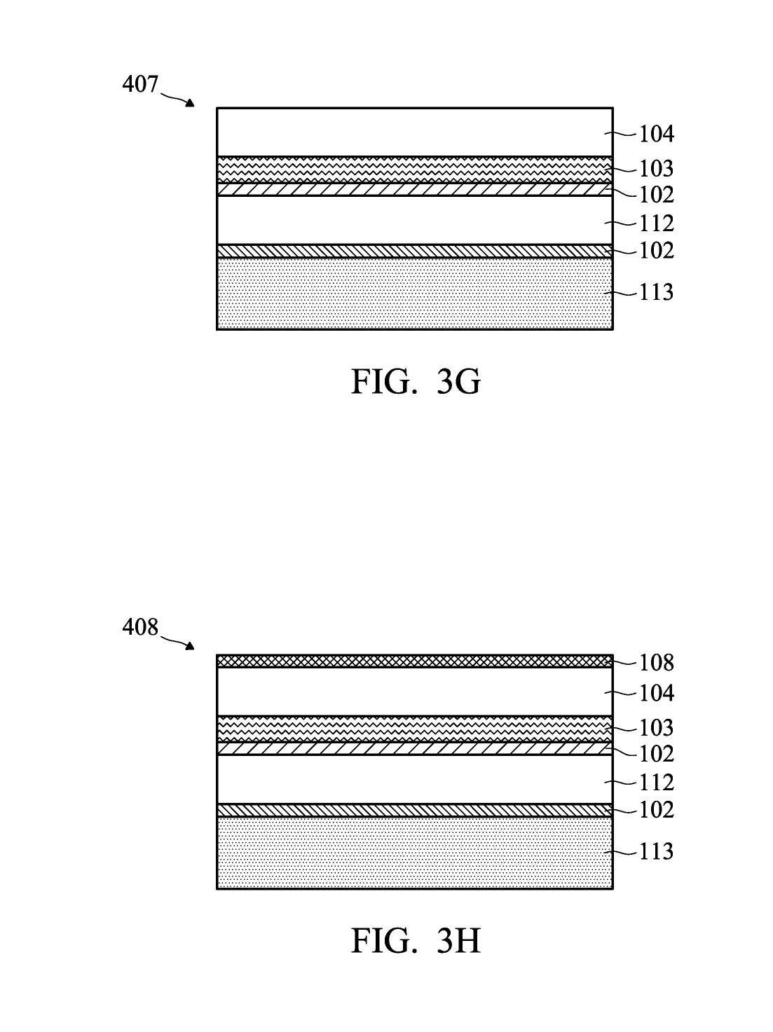

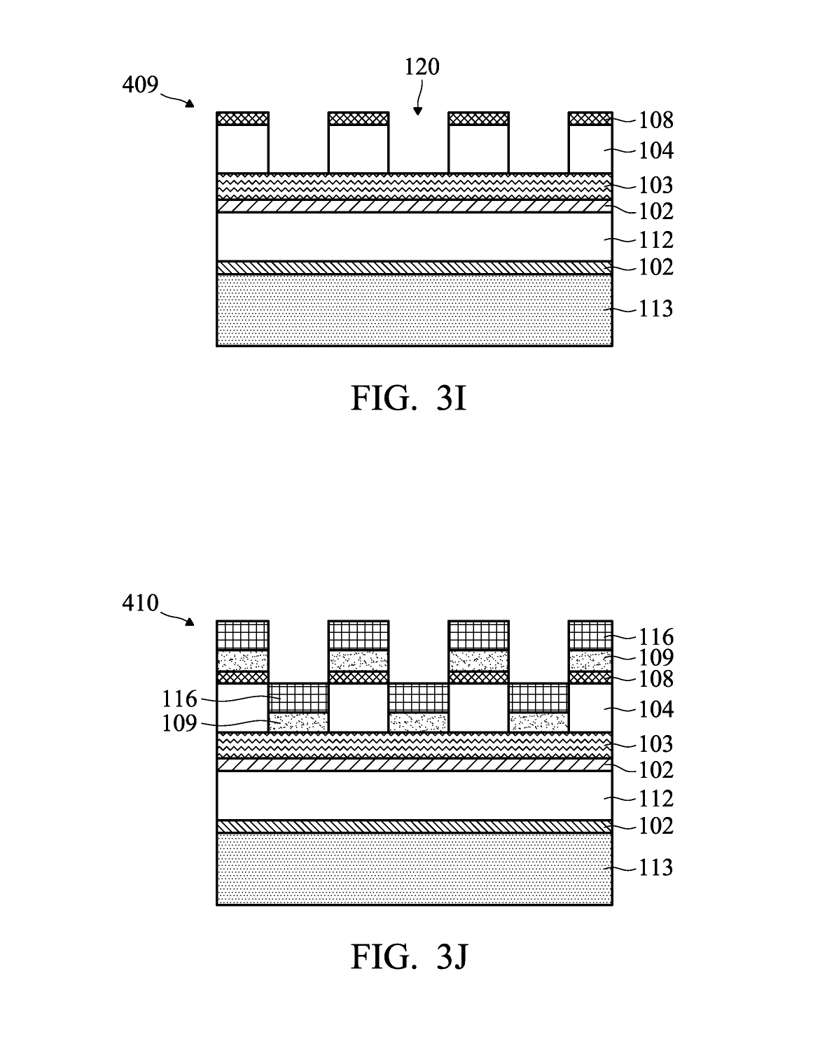

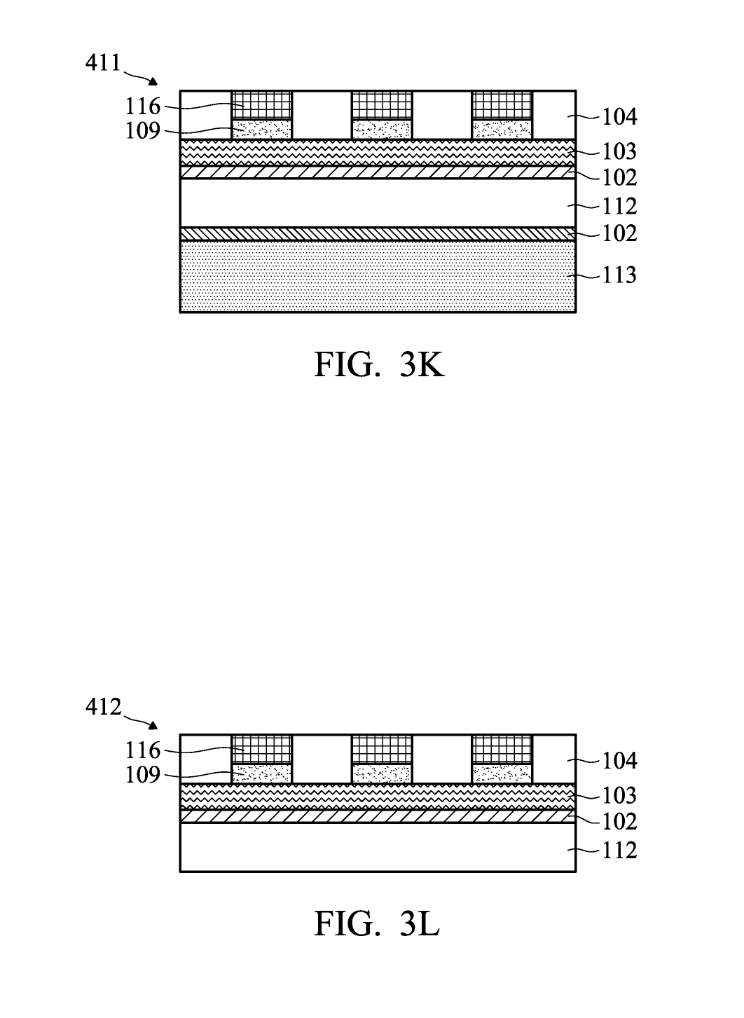

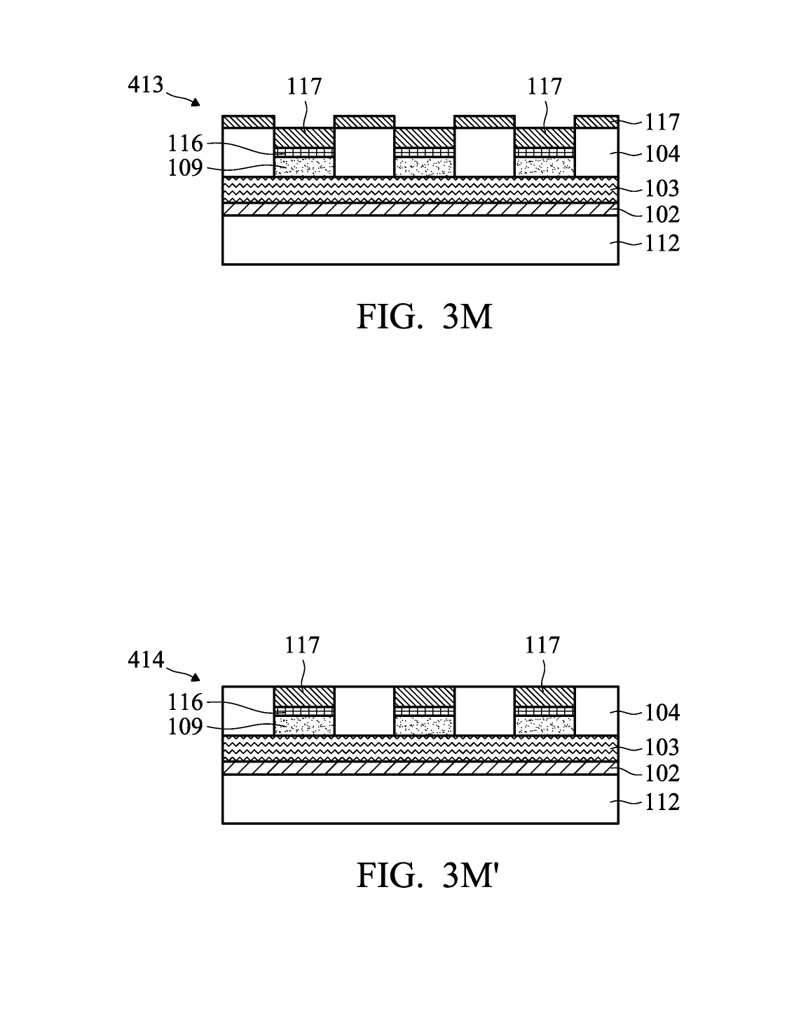

The third embodiment of the disclosure provides a process for fabricating a bio-functionalized and polymer-based microelectrode array of a subretinal prosthesis, in which the polymer is parylene-C. As shown in FIG. 3A˜FIG. 3M′, the flexible retinal prosthesis consisted of a flexible substrate with a plurality of photo sensors, microelectrodes, amplifiers, and microelectronic components, and it was made by a well-known technology such as the silicon-on-insulator (SOI) technology specialized for flexible integrated circuits. In brief, a circuit layer 103 was processed on a silicon wafer 101 with a silicon epitaxial layer 102 having 0.5-1.5 μm of thickness in Step 401 (FIG. 3A), using a well-known CMOS process technology. The circuit layer included a plurality of photo sensors, metal microelectrodes, amplifiers, microelectronic components and conductive traces which were specially designed for a subretinal prosthesis. A first parylene-C layer 104 having 10-15 μm of thickness was deposited on the silicon wafer 101 to cover the circuit layer 103 by using a chemical vapor deposition (CVD) process in Step 402 (FIG. 3B). Before the deposition of first parylene-C layer 104, an adhesion promoter (silane A-174) was used to improve the adhesion of first parylene-C layer 104 to the silicon nitride passivation layer on the surface of circuit layer 103. The silicon wafer substrate 101 was bonded to a first carrier substrate 111 on the surface of the first parylene-C layer 104 with a glue and a wafer bonding equipment in Step 403 (FIG. 3C). The first carrier substrate 111 was a silicon wafer with the same diameter and a silicon oxide layer 102 having 0.5-1.5 μm of thickness. Then, the silicon wafer 101 was mechanically thinned to a thickness of 40-60 μm by a wafer lapping equipment, followed by a silicon chemical etching process to remove the micro-crack resulting from the lapping process in Step 404 (FIG. 3D). The buried silicon epitaxial layer 102 was used for an etch stop. Subsequently, a second parylene-C layer 112 with the same 10 μm thickness as the first parylene-C layer 104 was deposited on the surface of the buried silicon epitaxial layer 102 in Step 405 (FIG. 3E). The adhesion promoter (silane A-174) was again vaporized onto the wafer surface before the deposition of a second parylene-C 112. A second carrier substrate 113 with a silicon oxide 102 layer having 0.5-1.5 μm of thickness was bonded to the silicon wafer substrate on the surface of the second parylene-C 112 with a glue and the same wafer bonding equipment in step 406 (FIG. 3F). The first carrier substrate 111 was then de-bonded from the silicon wafer substrate and the surface of the first parylene-C 104 was opened in Step 407 (FIG. 3G). Thereafter, a photoresist 108 was spun, exposed and developed on the surface of the first parylene-C 104 and was used as a masking material for defining the area of microelectrodes in Step 408 (FIG. 3H). The exposed parylene-C was then dry etched in O₂ plasma of 200 W, 50 sccm and a chamber pressure of 150 mTorr by reactive ion etching (RIE) in Step 409, and the area of electrode site 120 was opened (FIG. 3I). The opening area of the electrode sites 120 was 50×50 μm².

Titanium (Ti) (50-100 nm thickness), gold (400-1500 nm thickness), and titanium (50-100 nm thickness) layers were consecutively deposited on the parylene-C layer 104 in a sputter chamber with an argon (Ar) atmosphere of less than 0.005 mTorr in Step 410 (FIG. 3J). After the deposition of the Ti/Au/Ti layer 109, oxygen (O₂) was then introduced into the chamber. An IrOx layer 116 with 300-800 nm of thickness was sputtered onto the Ti/Au/Ti layer 109 in Ar/O₂ plasma using an RF magnetron sputter. Sputtering was performed with 1-2 second pulses followed by 5-10 second cool-down times to maintain low parylene-C temperatures. The IrOx layer 116 was patterned by a lift-off process in Step 411, using previously defined photoresist patterns. (FIG. 3K). A set of microelectrode stimulation pads with a geometric surface area of 1 mm², traces, and bonding pads on the electrode were fabricated as continuous titanium and iridium oxide layers on the circuit layer 103. The second carrier substrate 113 was de-bonded from the silicon wafer substrate and diced by using a femtosecond laser in Step 412 (FIG. 3L). The flexible retinal prosthesis had a flexible integrated circuit with a plurality of photo sensors and microelectrodes, encapsulated by two transparent parylene-C layers 104 and 112.

The bioactive molecules 117 as L1CAM was immobilized on the surface of IrOx 116 by piezoelectric dispensing technologies, or immobilized on the surface of IrOx 116 and parylene-C 104 by direct coating in Step 413 and 414 (FIG. 3M and FIG. 3M′). In brief, the surface of parylene-C 104 was first cleaned and roughened by a 200 mTorr oxygen plasma with 30 W power for 30 seconds. Immediately after the oxygen plasma treatment, a solution of a predetermined amount of bioactive molecules 117 as L1CAM was added onto the surface of the parylene-C layer 104, and it was placed inside an oven at 37° C. for 2 hours to allow for the immobilization of bioactive molecules 117 onto the surface of IrOx electrode layer 116 and parylene-C layer 104. The surface of IrOx electrode layer 116 and parylene-C layer 104 was washed with the phosphate buffer solution to remove unbounded bioactive molecules.

### Example 1

### Immobilization of Bioactive Molecules on the Surface of Parylene-C Substrate

To confirm the efficiency of bioactive molecules as being L1CAM immobilized onto the surface of the parylene-C substrate, an immunofluorescence staining (IFS) method was used to validate the immobilized bioactive molecules and a bicinchoninic acid (BCA) protein analysis was used to measure the amount of immobilized bioactive molecules. Laminin and/or collagen were set as comparative examples. It has previously been reported that Laminin and collagen can be immobilized on an electrode surface to improve the attachment and growth of neural cells.

Briefly, in the IFS method, the L1CAM-immobilized parylene-C substrate was incubated for 45 minutes in 5% bovine serum albumin (BSA) in a phosphate buffer solution to block a non-specific antibody, and reacted with human L1CAM polyclonal antibody (R&D, No. AF277) for 60 minutes. Subsequently, the substrate was stained with Fluorescein (FITC)-conjugated AffiniPure Rabbit Anti-goat IgG(H+L) (Jackson ImmunoResearch Laboratories, Inc.) for 60 minutes, washed with phosphate buffer, and covered with a glass slide. The efficiency of the immobilization of laminin and collagen I/III onto the surface of the parylene-C substrate was also tested. Digital images of the stained substrate were taken using a fluorescence microscope (Leica-090-135.002).

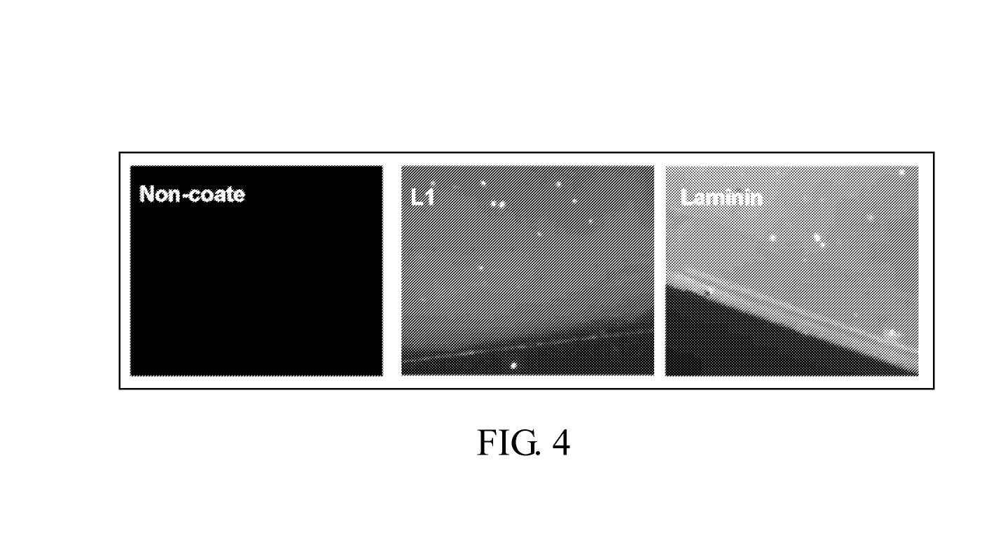

The result shown in FIG. 4 confirmed the immobilization of L1CAM and laminin onto the surface of the parylene-C substrate.

For the BCA protein analysis, the LavaPep peptide quantification kit (Gel Company, No. LP-022010) was used to measure the amount of immobilized bioactive molecules. As shown in TABLE 1, the molecular amounts of the immobilized L1CAM, laminin and collagen I/III onto the surface of the parylene-C substrate were equal, measured as 1.2×6.02×10e11, 1.5×6.02×10e11 and 5.9×6.02×10e11.

TABLE 1
BCA analysis of molecular numbers of biological
proteins immobilized on the parylene-C substrate.

| Bioactive | Immobilization | Amount of |
|---|---|---|
| Molecule | Efficiency (%) | Immobilized Molecules |
| L1CAM | 12.7 ± 2.8 | 1.2 × 6.02 × 10e11 |
| Laminin | 16.4 ± 1.7 | 1.5 × 6.02 × 10e11 |
| Collagen I/III | 2.2 ± 0.2 | 5.9 × 6.02 × 10e11 |

### Example 2

### Inhibition of L1CAM-Immobilized Parylene-C Substrate on the Attachment and Growth of Gial Cells

The inhibiting ability of the L1CAM-immobilized parylene-C substrate against gliosis sheath can be demonstrated by culturing glial cells onto the L1CAM-immobilized parylene-C substrate.

In a cell culture assay, the MTT assay was used to assess the viability (cell counting) and proliferation of C6 glial cells. The MTT assay measured the activity of cellular enzymes that reduced the MTT dye (3-(4,5-Dimethylthiazol-2-yl)-2,5-diphenyl-tetrazolium bromide) of yellow color to the formazan dye of purple color in living cells. C6 glial cells (CCRC 60046) of 2×10⁵ cells in 200 μl/well were seeded and cultured on the surface of L1CAM-immobilized parylene-C substrate for 24 hours. 20 μl MTT reagent (Sigma, No. M5655) was added into 0.5 mg/ml and 180 μl culture medium. The mediums were individually cultured for 2 hours in a 37° C. incubator with 5% CO₂. After insoluble crystals were completely dissolved in DMSO, absorbance at 570 nm was measured by using an ELISA microplate reader.

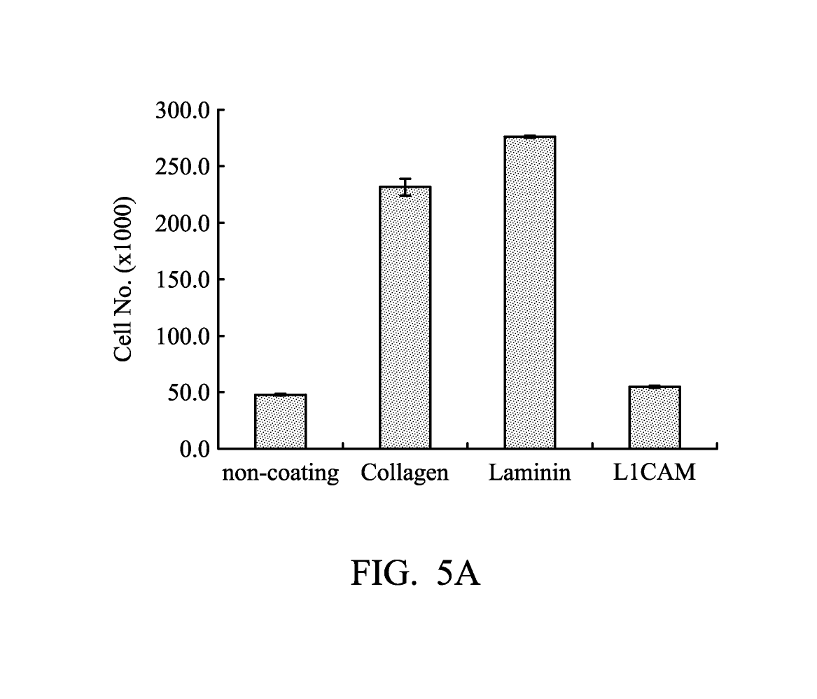

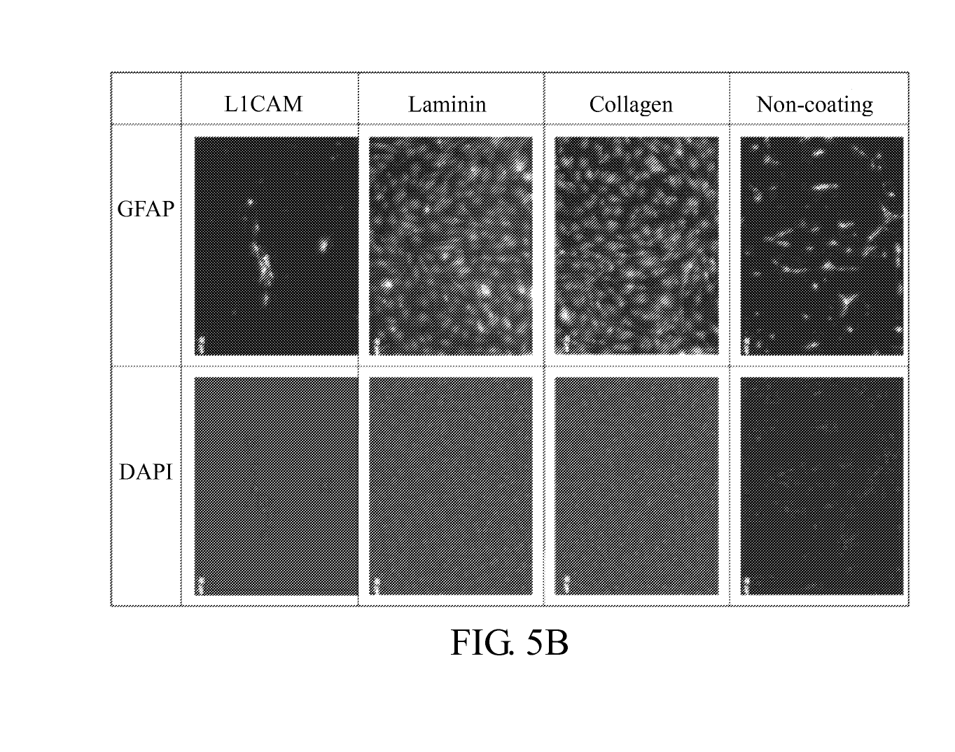

As shown in FIG. 5A, the attachment and proliferation of C6 glial cells on the L1CAM-immobilized parylene-C substrate were not observable, but the attachment of C6 glial cells on the lammin-immobilized parylene-C substrate and the collagen-immobilized parylene-C substrate were significantly enhanced.

The inhibiting ability of the L1CAM-immobilized parylene-C substrate against C6 glial cells was further verified by an immunofluorescence staining (IFS) method for glial fibrillary acidic protein (GFAP). GFAP was an intermediate filament protein that was thought to be specific for astrocytes. In brief, the sample was fixed with 4% paraformaldehyde (Sigma) for 60 minutes and twice rinsed with a phosphate buffer solution. 0.2% Triton X-100 (Sigma) was added. The sample was then incubated at room temperature for 5 minutes. Thereafter, the sample was treated with 5% bovine serum albumin (BSA) in a phosphate buffer solution to block a non-specific antibody, and reacted with polyclonal rabbit anti-GFAP (Dako#Z0334) for 60 minutes. The sample was stained with Fluorescein (FITC)-conjugated AffiniPure rabbit anti-goat IgG (H+L) (Jackson ImmunoResearch Laboratories, Inc.) for 60 minutes and washed with a phosphate buffer solution. The sample was then incubated with 0.1 μg/ml DAPI (4,6-diamidino-2-phenylindole) (Roche, No. 236276, nuclear staining) for 1 minute, washed with a phosphate buffer solution, and covered with a glass slide. The samples were mounted and observed by means of fluorescence microscopy (Leica-090-135.002).

The result shown in FIG. 5B confirmed that only a few C6 glial cells was attached on L1CAM-immobilized parylene-C substrate, but the attachment of C6 glial cells on the lammin-immobilized parylene-C substrate and the collagen-immobilized parylene-C substrate were significantly enhanced.

### Example 3

### L1CAM-Immobilized Parylene-C Substrate Selectively Promoting the Attachment and Outgrowth of Neural Cells

The ability of L1CAM-immobilized parylene-C substrate for selectively promoting neural cells attachment and neurite outgrowth to the surface of the substrate can be demonstrated by culturing neural cells onto the L1CAM-immobilized parylene-C substrate. Briefly, rat adrenal medulla pheochromocytoma PC12 neural cells (BCRC60048) were induced by culturing the cells in 100 ng/ml β-NGF for 48 hours. The resulting NGF-primed PC12 cells were seeded and cultured on the surface of L1CAM-immobilized parylene-C substrate for 24 hours followed by the addition of a 20 μl MTT reagent (0.5 mg/ml, Sigma, No. M5655) and 180 μl culture medium. The media were respectively cultured for 2 hours in a 37° C. incubator with 5% CO₂. After insoluble crystals were completely dissolved in DMSO, absorbance at 570 nm was measured by using an ELISA microplate reader.

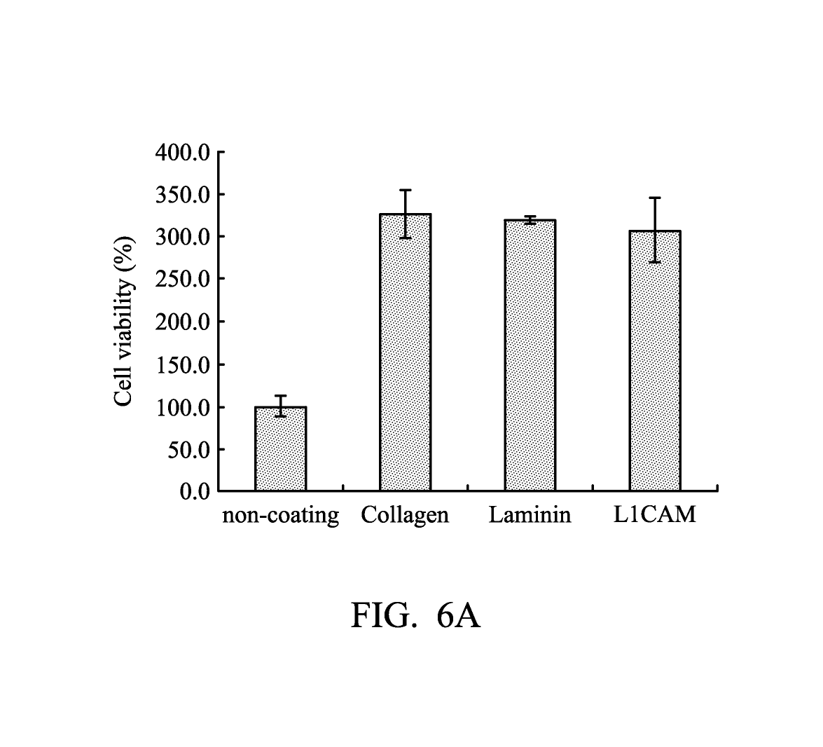

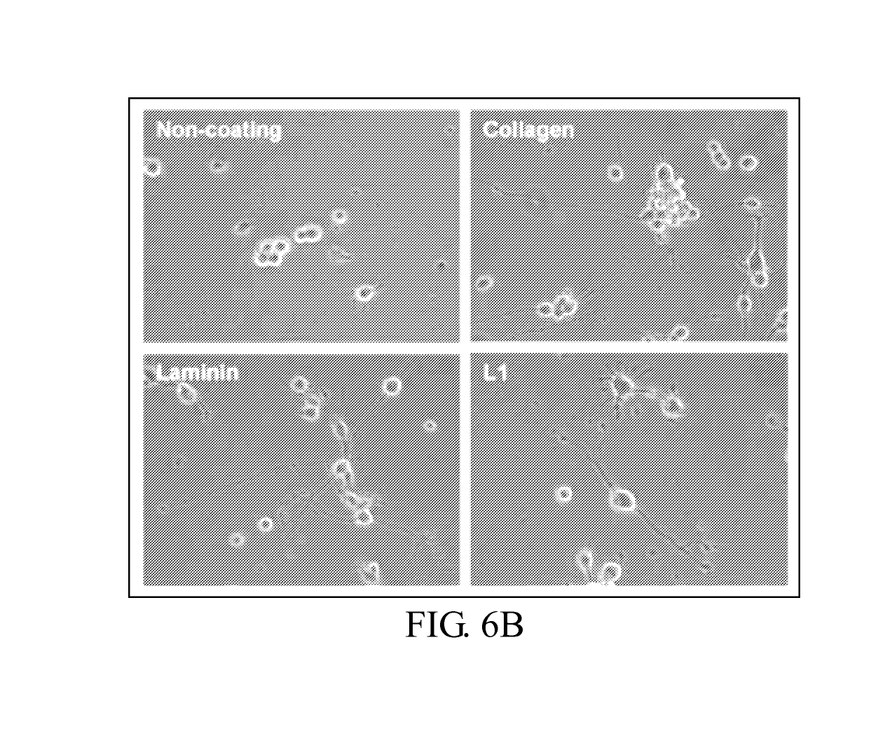

As shown in FIGS. 6A and 6B, the attachment and neurite outgrowth of NGF-primed PC12 cells on the L1CAM-immobilized parylene-C substrate were similar to that of laminin and collagen. These results demonstrate the ability of the L1CAM-immobilized surface to specifically promote neuronal growth and neurite extension, while inhibiting the attachment of astrocytes, one of the main cellular components of the glial sheath. Such unique properties present a vast potential to improve the biocompatibility and chronic performance of the microelectrode of retinal prostheses.

However, the unique property of L1CAM does not apply to all neural cells. The NT2 cell line, which is derived from a human teratocarcinoma, exhibits properties that are characteristic of a committed neuronal precursor at an early stage of development. NT2 cells can be induced by retinoic acid to differentiate in vitro into postmitotic central nervous system (CNS) neurons (NT2-N cells). The multi-potentiality and apparent functionality of the NT2 cell lines, have suggested that they may represent useful material for a variety of therapeutic approaches aimed at replacing dead neurons after neurodegenerative diseases or lesions of the CNS. It was found that L1CAM has no profound effect to promote the neuronal growth and neurite extension of a common neuron cell line NT2.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A retinal prosthesis, comprising
a microelectrode array, comprising a plurality of microelectrodes;
a polymer layer, partly encapsulating the microelectrode array, wherein the microelectrodes are exposed on the surface of the polymer layer; and
a layer of bioactive molecules, immobilized on the surface of the microelectrode.

2. The retinal prosthesis as claimed in claim 1, wherein the layer of bioactive molecules is further immobilized on the surface of the polymer layer.

3. The retinal prosthesis as claimed in claim 1, wherein the bioactive molecule comprises L1 cell adhesion molecule (L1CAM).

4. The retinal prosthesis as claimed in claim 1, wherein the polymer layer comprises parylene-C or polyimide.

5. The retinal prosthesis as claimed in claim 1, wherein the thickness of the polymer layer is greater than 10 μm and less than 20 μm.

6. The retinal prosthesis as claimed in claim 1, wherein the microelectrode comprises a Ti/metal double-layer or a Ti/metal/Ti triple-layer.

7. The retinal prosthesis as claimed in claim 6, wherein the metal of the Ti/metal double-layer and Ti/metal/Ti triple-layer is selected from a group consisting of aluminum (Al), titanium (Ti), platinum (Pt), gold (Au) and the alloy thereof.

8. The retinal prosthesis as claimed in claim 6, wherein the thickness of the Ti/metal double-layer is greater than 250 nm and less than 1 μm.

9. The retinal prosthesis as claimed in claim 6, wherein the thickness of Ti/metal/Ti double-layers layer is greater than 300 nm and less than 1 μm.

10. The retinal prosthesis as claimed in claim 6, wherein the Ti/metal double-layer or a Ti/metal/Ti triple-layer is sputtered with an iridium oxide (IrOx) layer (x≦4).

11. The retinal prosthesis as claimed in claim 10, wherein the thickness of IrOx layer is greater than 100 nm and less than 1 μm.

12. The retinal prosthesis as claimed in claim 1, wherein the microelectrode array further comprises a plurality of photo sensors, an integrated circuit and a power receiver.

13. The retinal prosthesis as claimed in claim 12, wherein the photo sensor is for receiving the optical imaging signal and for converting the optical imaging signal into the electrical signal.

14. The retinal prosthesis as claimed in claim 12, wherein the integrated circuit is in connecting with the microelectrodes and the photo sensors to receive the electrical signal from each photo sensor, amplify the electrical signal into the stimulation signal, and send the stimulation signal to the microelectrode.

15. The retinal prosthesis as claimed in claim 12, wherein the power receiver is within the substrate to receive energy from an external power source, transform the energy into an electrical power, and send the electrical power to the integrated circuit.

16. The retinal prosthesis as claimed in claim 1, comprising epiretinal prosthesis or subretinal prosthesis.

17. A method for the treatment of degenerative retinal diseases, comprising applying the retinal prosthesis as claimed in claim 1 to the retina of a subject in need.

18. The method as claimed in claim 17, wherein the degenerative retinal disease comprises glaucoma, retinitis pigmentosa (RP) or age-related macular degeneration (AMD).

19. A method for the treatment of degenerative retinal diseases, comprising applying the retinal prosthesis as claimed in claim 2 to the retina of a subject in need.

20. The method as claimed in claim 19, wherein the degenerative retinal disease comprises glaucoma, retinitis pigmentosa (RP) or age-related macular degeneration (AMD).
